(19) United States
(12) Patent Application Publication
(10) Pub. No.: US 2016/0128556 A1
(43) Pub. Date: May 12, 2016

(54) VIDEO LARYNGOSCOPE

(71) Applicant: Hsien-Yung LAI, Hualien County (TW)

(72) Inventor: Hsien-Yung LAI, Hualien County (TW)

(21) Appl. No.: 14/937,901

(22) Filed: Nov. 11, 2015

(30) Foreign Application Priority Data
Nov. 12, 2014 (TW) 103139267

(51) Int. Cl. A61B 1/267; A61B 1/00; A61B 5/00; A61B 1/05

(52) U.S. Cl. CPC A61B 1/267; A61B 1/051; A61B 1/00124; A61B 1/00165; A61B 5/6898

(57) ABSTRACT

A video laryngoscope includes a handle, a blade, a lens, a photo sensor, and an output terminal. The blade is connected to the handle. The lens is disposed on the blade. The photo sensor is connected to the lens to transform an image obtained by the lens into an electrical signal. The output terminal is disposed on the handle and is electrically connected to the photo sensor. The output terminal is configured to allow a smart communication device to be connected thereto, such that the electrical signal can be output to the smart communication device through the output terminal.

### CROSS-REFERENCE TO RELATED APPLICATION

This application claims the priority benefit of Taiwanese application serial no. 103139267, filed Nov. 12, 2014, the full disclosure of which is incorporated herein by reference.

### BACKGROUND

1. Technical Field

This disclosure relates to laryngoscopes. More particularly, the disclosure relates to video laryngoscopes.

2. Description of Related Art

Endotracheal intubation is inserting an endotracheal tube from a mouth or a nasal cavity, through the throat and the glottis, and into the trachea, whereby an artificial open airway is established. Common causes of intubation include respiratory failure and respiratory track having no self-protection. In early times, endotracheal intubation is performed by fingers groping. Latter, direct laryngoscope began to be widely used. Recently, videos through optical fibers were added into the direct laryngoscopes to become video laryngoscopes. This technology improves the past problem of difficult intubation due to that the glottis entrance cannot be seen. At the same time, the infection problem caused by medical personnel closing to the nose and mouth of the patient can be avoided.

However, most of the present video laryngoscope can only display images on screen, but cannot access the images. Hence, the images cannot be edited, copied, or transferred. Accordingly, the video laryngoscopes still have limitations in applications to be solved.

### SUMMARY

According to some embodiments, a video laryngoscope includes a handle, a blade, a lens, a photo sensor, and an output terminal. The blade is connected to the handle. The lens is disposed on the blade. The photo sensor is connected to the lens to transform an image obtained by the lens into an electrical signal. The output terminal is disposed on the handle and is electrically connected to the photo sensor. The output terminal is configured to allow a smart communication device to be connected thereto, such that the electrical signal can be output to the smart communication device through the output terminal.

### BRIEF DESCRIPTION OF THE DRAWINGS

FIG. 1 is a perspective view of a video laryngoscope according to some embodiments of the present disclosure.

FIG. 2 is an exploded view of the video laryngoscope in FIG. 1.

FIG. 3 is a functional block diagram of the video laryngoscope in FIG. 1.

FIG. 4 is a perspective view of a video laryngoscope according to some other embodiments of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. in other instances, well-known structures and devices are schematically shown in order to simplify the drawing.

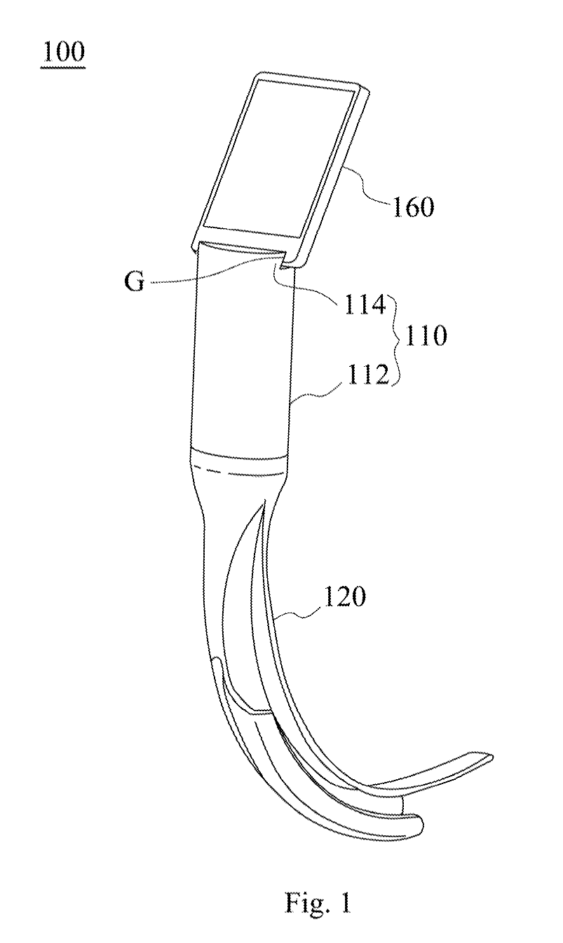

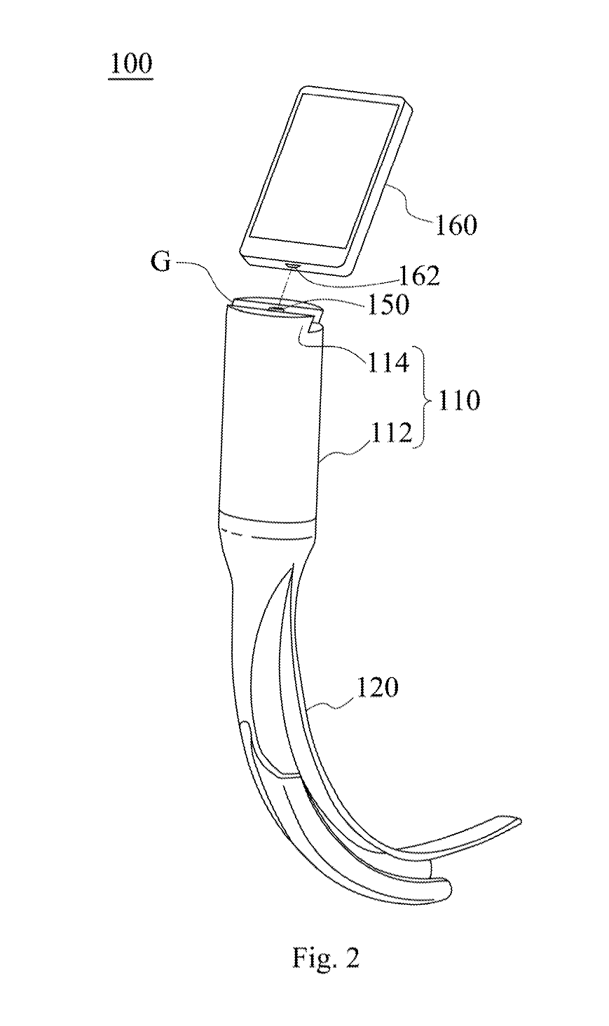

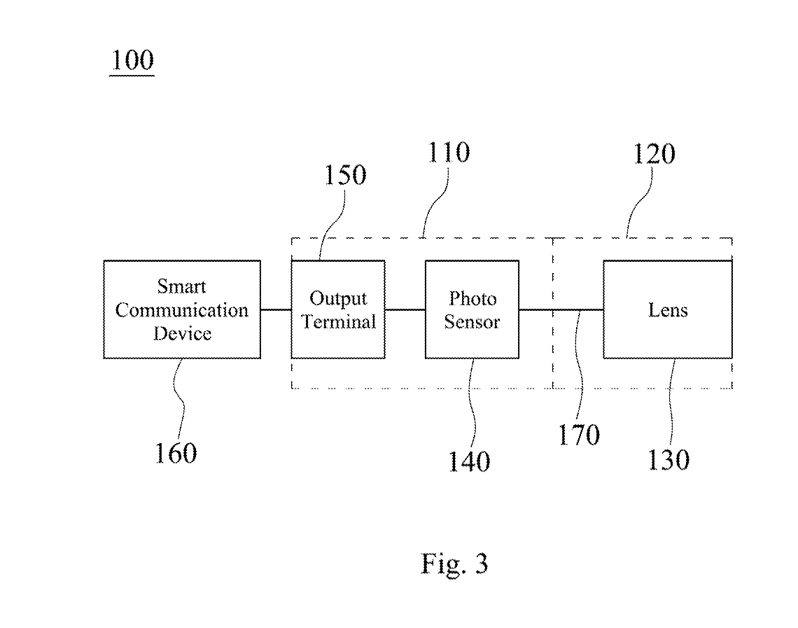

FIG. 1 is a perspective view of a video laryngoscope 100 according to some embodiments of the present disclosure. FIG. 2 is an exploded view of the video laryngoscope 100 in FIG. 1. FIG. 3 is a functional block diagram of the video laryngoscope 100 in FIG. 1. As shown in FIGS. 1-3, a video laryngoscope 100 includes a handle 110, a blade 120, a lens 130, a photo sensor 140, and an output terminal 150. The blade 120 is connected to the handle 110. The lens 130 is disposed on the blade 120. The photo sensor 140 is connected to the lens 130 to transform an image obtained by the lens 130 into an electrical signal. The output terminal 150 is disposed on the handle 110 and is electrically connected to the photo sensor 140. The output terminal 150 is configured to allow a smart communication device 160 to be connected thereto, such that the electrical signal can be output to the smart communication device 160 through the output terminal 150.

Since the electrical signals transformed from the images obtained by the lens 130 will be output to the smart communication device 160 the smart communication device 160 can be used to display the images of the lens 130 to achieve the function of the video laryngoscope 100. Moreover, these images also can be stored in the smart communication device 160 when needed, and can be used by physicians or hospital for subsequent records or edit. Furthermore, in teaching, teachers only need to use the smart communication device 160 to transfer the images to students, and then the students can display the images through their own electronic devices. Therefore, a large group of people huddled together to see teacher's demonstration is not needed.

The handle 110 includes a main body 112 and a position fixing feature 114. The position fixing feature 114 is disposed on the main body 112 for temporarily fixing the smart communication device 160 on the main body 112. The meaning of “temporarily fixing the smart communication device 160 on the main body 112” should be explained that “the smart communication device 160 is fixed to the main body 112, and no damage will be caused to both the smart communication device 160 and the main body 112 when the smart communication device 160 is separated from the main body 112 by a user.”

More specifically, the position fixing feature 114 can be a tool-free position fixing feature. For example, the position fixing feature 114 may include a pair of bumps. The pair of the bumps defines an interval G therebetween. The smart communication device 160 can be at least partly fastened in the interval G. Therefore, the relative positions of the smart communication device 160 and the main body 112 can be temporarily fixed, and the smart communication device 160 and the main body 112 can be then separated by a user without using any tool. It should be understood that the position fixing feature 114 is only an exemplary embodiment, other various tool-free position fixing features, such as a chute, leaf spring, hook, pin etc., can also be used. Persons having ordinary skills in the art can flexibly choose the position fixing feature 114 depending on the actual needs.

Furthermore, in some embodiments, the output terminal 150 can be positioned in the interval G. When the smart communication device 160 is temporarily fixed to the main body 112 by the position fixing feature 114, the output terminal 150 can be just connected to the connection port 162 of the smart communication device 160, whereby the output terminal 150 and the connection port 162 can be more easily connected by a user.

In some embodiments, the position fixing feature 114 is disposed on a terminal of the main body 112 away from the blade 120. Therefore, the smart communication device 160 would exactly face the user to facilitate the user's observation when the smart communication device 160 is temporarily fixed to the main body 112 by the position fixing feature 114.

In some embodiments, the photo sensor 140 is accommodated in the handle 110. That is, the handle 110 has a cavity, and the photo sensor 140 can be accommodated in the cavity of the handle 110. Furthermore, when the electricity of the video laryngoscope 100 is from batteries, such as rechargeable batteries or primary batteries, the batteries can also be installed in the handle 110. Certainly, the electricity of the video laryngoscope 100 can also be from mains electricity. When the electricity of the video laryngoscope 100 is from mains electricity, a power cable can also be coupled with the handle 110.

Practically, the photo sensor 140 may be a charge-coupled device (CCD), active-pixel sensor (APS), complementary metal-oxide-semiconductor image sensor (CMOS), or any combinations thereof.

Moreover, the video laryngoscope 100 further includes an optical fiber 170 connecting the lens 130 with the photo sensor 140, and the optical fiber 170 is partially accommodated in the blade 120. More specifically, the blade 120 may have a channel therein. The optical fiber 170 couples the lens 130 with the photo sensor 140 through the channel of the blade 120.

The output terminal 150 may use a video transmission interface in line with the common specifications. The meaning of “the common specifications” is “compatible with at least two different brands and/or models of smart communication devices.” For example, the output terminal 150 may be a mobile high-definition link (MHL) interface. Since most of the present smart communication devices support MHL interface, the output terminal 150 may use MHL interface being compatible with most commercial smart communication devices. Certainly, other suitable video transmission interface, such as high definition multimedia interface (HDMI), can also be used as the output terminal 150. Persons having ordinary skills in the art can flexibly choose the output terminal 150 depending on the actual needs.

Practically, the smart communication device 160 may be any electronic devices having functions of communication, access, and calculation. For example, the smart communication device 160 may be a smart phone, personal digital assistant (PDA), tablet computer, electronic dictionary, ebook reader, notebook computer, computer, or any combinations thereof.

In some embodiments, the output terminal 150 is directly connected with the smart communication device 160. That is, the smart communication device 160 is directly connected to the output terminal 150 and temporarily fixed on the main body 112, and no other cable is between the smart communication device 160 and the output terminal 150. This configuration can make the entire device more simple, and only one element of the video laryngoscope 100 is needed to be held when operating, no other component is needed to fix the smart communication device 160.

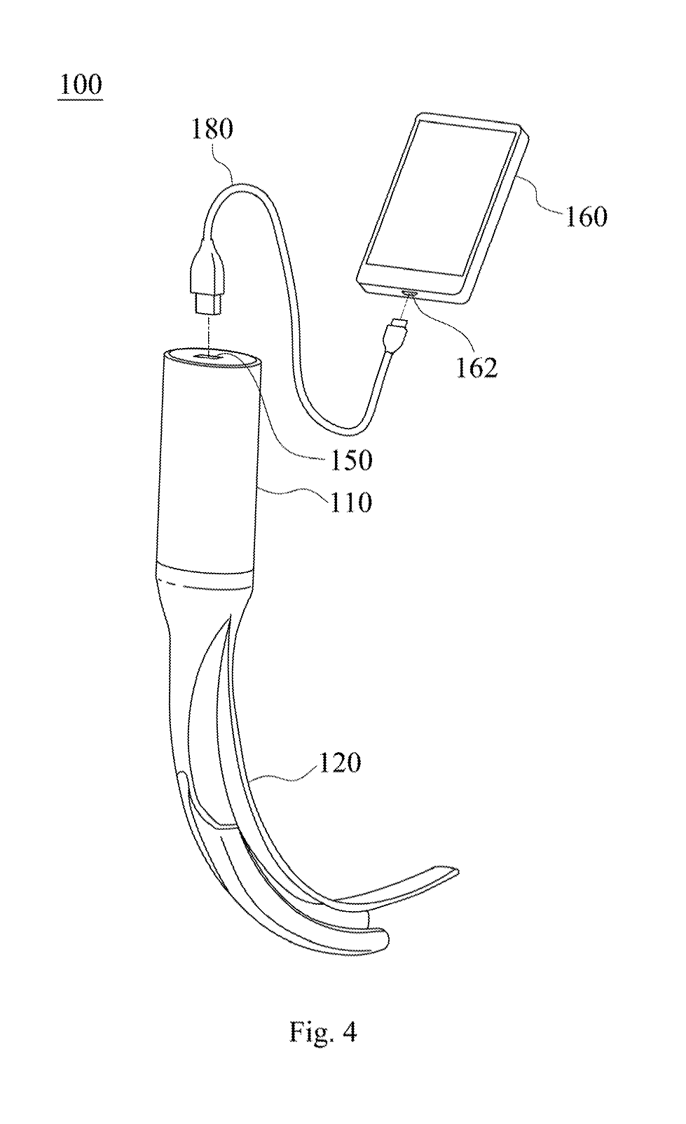

FIG. 4 is a perspective view of a video laryngoscope 100 according to some other embodiments of the present disclosure. The difference between FIG. 4 and FIG. 1 is the video laryngoscope 100 of FIG. 4 further includes a connecting wire 180. The connecting wire 180 couples the output terminal 150 with the connection port 162 of the smart communication device 160. That is, in some embodiments, the output terminal 150 is also not directly connected to the smart communication device 160, and the smart communication device 160 is not temporarily fixed to the main body 112. Persons having ordinary skills in the art can flexibly choose the connecting way between the output terminal 150 and the connection port 162 of the smart communication device 160 depending on the actual needs.

Other related structural details are all the same as the previous embodiments, and thus are omitted here.

What is claimed is:

1. A video laryngoscope, comprising:
a handle;
a blade connected to the handle;
a lens disposed on the blade;
a photo sensor connected to the lens for transforming an image obtained by the lens into an electrical signal; and
an output terminal disposed on the handle and being electrically connected to the photo sensor, wherein the output terminal is configured to allow a smart communication device to be connected thereto, such that the electrical signal can be output to the smart communication device through the output terminal.

2. The video laryngoscope of claim 1, wherein the handle comprises:
a main body; and
at least a position fixing feature disposed on the main body for temporarily fixing the smart communication device on the main body.

3. The video laryngoscope of claim 2, wherein the position fixing feature is a tool-free position fixing feature.

4. The video laryngoscope of claim 2, wherein the output terminal is connected to the smart communication device when the smart communication device is temporarily fixed on the main body by the position fixing feature.

5. The video laryngoscope of claim 2, wherein the position fixing feature is disposed on a terminal of the main body away from the blade.

6. The video laryngoscope of claim 1, wherein the output terminal is disposed on a terminal of the main body away from the blade.

7. The video laryngoscope of claim 1, wherein the photo sensor is accommodated in the handle.

8. The video laryngoscope of claim 1, further comprising:
an optical fiber connecting the lens with the photo sensor, wherein the optical fiber is partially accommodated in the blade.

9. The video laryngoscope of claim 1, wherein the output terminal can be directly connected to the smart communication device.

10. The video laryngoscope of claim 1, further comprising:
a connecting wire being capable of coupling the output terminal with the smart communication device.
